(19) United States
(12) Patent Application Publication
(10) Pub. No.: US 2012/0039796 A1
(43) Pub. Date: Feb. 16, 2012

(54) Novel method for creating, suspending and stabilizing electronically modified oxygen derivatives, along with creating, suspending and stabilizing electronically modified reaction intermediates, in a bio compatible fluorocarbon suspension, for the purpose of inducing a cascading immune response in mammalian patients

(76) Inventor: Demetrios Markou, Lemont, IL (US)

(21) Appl. No.: 13/209,369

(22) Filed: Aug. 13, 2011

Related U.S. Application Data
(60) Provisional application No. 61/373,836, filed on Aug. 15, 2010.

(51) Int. Cl. A61K 51/00; A61K 33/26; A61K 33/44; A61K 38/43; A61K 31/7105; A61K 31/7088; A61K 31/05; A61K 31/122; A61K 31/192; A61K 31/385; A61K 33/04; A61K 31/352; A61K 31/12; A61K 36/00; A61K 31/711; A61K 31/7052; A61K 31/015; A61K 31/685; A61K 31/66; A61K 35/54; A61K 31/205; A61K 38/02; A61K 9/00; A61P 35/00; A61P 37/04; A61K 33/24

(52) U.S. Cl. 424/1.11; 424/649; 424/646; 424/125; 424/94.1; 514/43; 514/44 R; 514/731; 514/734; 514/688; 514/570; 514/440; 424/702; 514/457; 514/685; 514/22; 514/682; 514/455; 514/764; 514/78; 514/120; 424/581; 514/554; 514/456; 514/733; 514/1.1; 424/484

(57) ABSTRACT

A bio compatible free radical suspension, comprising of oxygen and electronically modified reaction intermediates, where a fluorocarbon is used as an inert medium for stabilization of reaction intermediates. A stabilized bio compatible electronically modified derivative suspension is produced by the subjecting a fluorocarbon to certain stressors, such as oxidizing agents, reactive intermediates, physiological gases, benzo-γ-pyrone derivatives, ultrasonic-cavitation, electric fields, magnetic fields, UV radiation, active metal catalyst, surfactant reactants, buffers, electrolytes, glucose, glucose derivatives, for the purpose of inducing a cascading immune response.

### CROSS-REFERENCE TO RELATED APPLICATIONS

This application claims priority to the following: U.S. Provisional Patent Application No. 61/373,836, filed on Aug. 15, 2010.

### DESCRIPTION OF RELATED ART

U.S. Pat. No. 5,869,539, issued Feb. 9, 1999, to Garfield, et al. Titled “Emulsions of perfluoro compounds as solvents for nitric oxide (NO)” U.S. Pat. No. 5,869,539 teaches us that NO is relatively a stable molecule when not in the presence of oxygen. Nitric oxide (NO) is a gas with low solubility in water and aqueous solutions such as serum. The author states although NO is considered to be a free radical, it is so in the sense that oxygen is considered a free radical, which it is a stable di-radical, NO is an uncharged radical like oxygen, thus making NO stable enough not to interact chemically with biological fluids or with most organic solvents, unlike Ozone and the charged reactive intermediates in this invention. The gases; nitrogen, oxygen, and nitric oxide, have diatomic molecules and similar molecular weights. The first two, however, are non-polar molecules, and therefore have slightly lower solubility's in water than NO. The volatilities of these three gases are also fairly similar, but the polarity of the NO molecule makes it the least volatile, as seen from the boiling points at normal pressure: −195 degree, −183 degree, and −151 degree C.

“Nitric oxide reacts instantaneously with oxygen from air, yielding nitrogen dioxide, a toxic red-brown gas. Therefore, all studies with NO must be carried out in the absence of oxygen or oxidizing media” 2 NO+O₂→2 NO₂. Nitric oxide reacts with oxygen and water to form nitrous acid: HNO₂, 4 NO+O₂+2H₂O→4 HNO₂.

Nitrogen oxides of biologic relevance include elemental nitrogen have five oxidation states (NOₓ: N₂O, NO., NO₂⁻, NO₂., NO₃⁻). NO is one of the biologically active Nitrogen oxides. Therefore, NO does not remain as NO.-radical moiety in biological environments, in aqueous systems and at air-liquid interfaces, NO.-generation yields nitrite (NO₂⁻) and nitrate (NO₃⁻) as end products. The NO.-radical reacts rapidly with the superoxide radical, forming highly reactive peroxynitrite anion (ONOO⁻).

One aspect of my invention uses a PFC as a dielectric, were one can electronically modify molecules to produce reaction intermediates when an applied voltage is added, it might be useful to study the oxidation states of NO in a laboratory setting. Though Uncharged NO is not within scope of this invention but NO's charged electronically modified reaction intermediates can be.

U.S. Pat. No. 6,399,664 issued Jan. 4, 2002 to Smith.

Titled “Method of treating cancer, specifically leukemia, with ozone”

“The invention relates to a method for treatment of leukemia in mammals, and more specifically chronic myeloid leukemia (CML), using reactive oxygen intermediates. Reactive oxygen intermediates are administered in a therapeutically effective amount to a mammal that has leukemia. The administration of reactive oxygen intermediates, and more specifically ozone, has been found to be particularly effective in the treatment of CML and on the modulation of the immune and hematopoietic systems of mammals having cancer.”

Where the author teaches us that “(a) direct injection of said ozone into said mammal; (b) ex vivo treatment of blood from said mammal with said ozone followed by reinfusion of said treated blood into said mammal; (c) injection of ozone treated products into said mammal; (d) inhalation of said ozone treated products; (e) insufflations of said ozone”

My invention solves the deficiencies of this method, not only can you get rid of the expensive machines on site, such as dialysis ozone reinfusion machines, the practitioner does not need to be exposed to a patient's blood and risk cross contamination. Synthetic bio compatible PFC's offer greater flexibility, and what you can do with a synthetic delivery system is include other drugs or compounds within the solution to act in a synergistic manner and increase the probability of a successful outcome.

U.S. Pat. No. 6,537,380 issued Mar. 25, 2003 to Zazerra, et al.

Titled “Fluorinated solvent compositions containing ozone”

Where the author teaches us a method of cleaning a silicon substrate comprising contacting said substrate with a composition comprising ozone and a fluorinated solvent. The author has a wide scope of fluorinated solvents that can be used, though the fluorinated solvents particularly useful to form the stable composition of the invention comprises of hydrofluoroethers, with extremely low surface tension with quick evaporation properties for cleaning electronic substrates, specifically silicon, polysilicon, silicon oxides and microelectro-mechanical devices. HFE compounds due to their properties are idea for cleaning electrical devices and silicon wafers. The invention described a cleaning composition useful for oxidation of a substrate, removal of residues, rinsing and drying, and has an efficient rate of surface oxidation. The author indicates in the invention it is preferred that the fluorinated solvent be partially or incompletely fluorinated. Though ozone is added to the enhance the industrial cleaning abilities of the hydrofluoroether, The C—H bond in hydrofluorocarbons, are not resistant to oxidizing agents, it has been suggested the harmful effects of chlorofluorocarbon compounds (CFCs) on the ozone layer in the atmosphere has led to the search for more environmentally acceptable alternatives. Hydrofluoroethers (HFEs) have been proposed as CFC replacements. One advantage of these compounds is the presence of C—H bonds which allows them to be attacked by OH radicals and there by undergo reactions in the troposphere. The hydrogen bond is susceptible to attack from strong oxidizing agents.

My invention teaches us, how to store, stabilize and deliver electronically modified oxygen derivatives, along with reaction intermediates such as benzo-γ-pyrone derivatives in a bio compatible PFC matrix, were once introduced to a mammalian body induces an immune response. The preferred Hydro-fluorinated substances of invention U.S. Pat. No. 6,537,380 are sold from the 3m company under the brand name HFE-7100 are specifically well suited for cleaning electronic devices, the industrial fluorinated compounds sold from 3m are not bio compatible and are not suited to be used in a mammalian body. The preferred fluorocarbon in the invention is not suitable to stabilize and store ozone due to the hydrogen bond, it should be noted that the hydro fluorinated ethers used in invention U.S. Pat. No. 6,537,380 pose serious health risks if inhaled and may cause death.

The utility of my invention is the ability to store and stabilize electronically modified reaction intermediates in a bio compatible PFC matrix. My invention specifically solves the short comings in modern Ozone therapy; I am able to show through repeatable experimental evidence that stabilization of EMODs is a reality. Whereas before this present invention one must deliver EMODs to a patient on site before the rapid decay of the molecule, and in a concentration that is not effective due to solubility factor. You are now able to store highly reactive electronically modified intermediates in the short term and long, another aspect of my invention is where ozone and oxygen is used to drive reaction's with in a bio compatible PFC solution and yet another embodiment of my invention is where a PFC is used as a dielectric with an applied voltage, to electronically modify compounds within the PFC matrix, were these compounds are stabilized within the inert PFC matrix. EMODs controls apoptosis of a cell and in stabilizing these reaction intermediates in a bio compatible PFC in essence creates a new drug for therapeutic uses; I cannot stress the benefit of this discovery to humanity and mammalian patients worldwide from regeneration of ligaments to apoptosis of cancer, to deactivating viruses.

U.S. Pat. No. 4,497,829 issued Feb. 5, 1985 to sloviter, et al. Titled “Process for preparing a perfluorchemical emulsion artificial blood”

U.S. Pat. No. 4,497,829 teaches us on how to make a stable emulsion using sonication, in this invention the author uses the emulsified fluorocarbon as an artificial blood substitute, a composition containing oxygen, emulsified in a physiologically acceptable aqueous medium, the perfluorochemical particles being coated with a lipid which is non-antigenic. The preferred lipid is a phospholipid such as lecithin, available in the form of egg yolk phospholipid. Lecithin is also present in soybean phospholipid. The perfluoro compound emulsion of the present invention is prepared by sonicfication and further centrifuged were Large particles are eliminated by discarding the bottom fraction of the emulsion, the author shows average particle size done in this manner is 0.1 um. The author further states this was the first stable emulsion of a single perfluorochemical useful as an artificial blood which is nonhemolytic, autoclavable by conventional techniques, and storable at normal refrigeration temperatures. The composition may even be stored at room temperatures for considerable periods. Finally, the present composition is both isotonic and isoionic with respect to natural blood plasma. This method is how most emulsions to date are created using ultrasonic cavitation technology to disperse immiscible substrates. There are only 2 methods one can make an emulsion, ultrasonic cavitation, and or through high-pressure homogenization a combination thereof.

U.S. Pat. No. 4,632,980 Zee et al. titled “Ozone decontamination of blood and blood products”

The author discloses a method of treating blood and blood products of enveloped viruses by contacting the blood or blood components in an aqueous medium by an amount of ozone administered in the blood, were it is in-fused back into the patient.

U.S. Pat. No. 6,569,467 Bolton issued on May 27, 2003, Titled “Treatment of autoimmune diseases”

The author describes a method were an autoimmune vaccine is created by subjecting the blood aliquot to ozone, UV radiation and elevated temperature, and rein infused back in to the body to alleviate the symptoms of autoimmune diseases such as rheumatoid arthritis. The vaccine comprises an aliquot of the patient's blood, containing, inter alia, leukocytes having up regulated expression of various cell surface markers and lymphocytes containing decreased amounts of certain stress proteins called heat shock proteins HSP.

U.S. Pat. No. 3,352,642 issued Nov. 14, 1967, to Heidt, Lawrence J.

Landi, Vincent R, Titled “stabilization of ozone”

The author teaches us ozone can be stable in a strong base of sodium hydroxide, were its stored in a container where the walls of the container is rendered inert by reacting them with NaOH.

United States Patent Application 20040254092 publication date Dec. 16, 2004 from Zhen-man, Lin Titled ‘Surface treatmeat of sars-infected lungs’

After reading patent application 20040254092 it was apparent the author was not skilled in the art of medical treatments or in methods using ozone in a bio compatible application. Though ozone is relatively safe and non toxic in blood applications, and has been used for a better than 100 years in various therapies with surprising results, the author described a method to treat a SARS infected lung, using ozone and a PFC solvent, where the solvent is pump into the lung via a supersonic pump generator, were the thought of the author is, the ozone pump in, will oxidize and deactivate the SARS virus. I am of the opinion that, there is no achievable concentration that can make such a therapy possible or safe, again no achievable concentration. As little as 2 to 5 parts per mill can damage DNA irreversible in the lung, and cause permanent scar formation, the powerful oxidizing action of ozone will irreversible destroy delicate sac sponge like alveoli tissue immediately on contact, especially in the concentration discussed in the application. Other PFCs mentioned for use for the invention was C5F9H30, this PFC can be disastrous, were on contact with ozone will attack the hydrogen bond and soon become acidic and eat the delicate tissues of the lung, not to mention the devastating hydroxyl radical cascade being formed once ozone enters the lung. It's clear the data the author presented was incomplete, and highly flawed, though ozone is known to deactivate viruses and that seems to be the original thought for inspiration of this application. This patent application has absolutely no utility in lung ozone exposure applications, and in my opinion is based on pure incomplete understanding of the biology, and a gross underestimation of the powerful oxidizing capabilities of ozone and oxygen in medical applications. A particular quote stood out from the author and sums up the thought process ‘I do not specialize in medicine but just a little medically minded.” That quote can sum up the application; the method the author describes will destroy the sensitive tissues of the lung, leaving devastating scar tissue, and can never be accepted as a real therapy. Now a method to treat SARS or any virus is not ozonate the lungs, one can always try IV ozone infusions at a qualified ozone clinic, to induce a cascading immune response, blood does flows to the capillaries that wrap around the alveoli sacs, where the air to blood exchange happens.

Ozone therapy as in IV infusions, topical uses, and as in prolo-therapy is being used all over the world today, ozone therapy has been used by hundreds of thousands of practitioners over the last 100 years, it's safe, effective but the draw backs till now, keeps it from becoming a main stream therapy, now prolo-ozone therapy can now become main stream or at least attract more practitioners to use because they do not need to make the investment in ozone machines, and even if they do, bubbling ozone in saline is almost futile. In ozone IV infusions, you do not need to remove blood from the patient any more. The utility of the patent is evident and need's patent protection, I am in the process of manufacturing, for use in a veterinary setting, were I will be shipping frozen samples packed in dry ice, for ligament prolo-therapy injections for animals, such as race horse, or just for pet use. And the emulsion formula will be used in a veterinary setting also, to gather data for further human trails. The utility in medical applications of my invention is enormous; you can now keep compounds in their intermediate form, bypassing key biological path ways that modern drugs must pass through to reach an end result.

### BACKGROUND

This invention is based on the stunning realization that some highly fluorinated perfluorocarbons such as perfluoroalkanes are inert to oxidizing agents; such as ozone, high energy uv radiation such as in the upper atmosphere, PFCs can be subjected to electric fields, and high temperature without breaking down up to 400 degree C. McElroy et al. investigated the atmospheric fate of various perfluorinated compounds including C6 to C10 perfluoroalkanes. They concluded that perfluorocarbons do not react at significant rates with hydroxyl radicals and that such compounds will only degrade in the upper atmosphere through reactions with O(1D) yielding an approximate average atmospheric lifetime of 1,000 years. More recent work at MIT has shown that perfluoroalkanes do not react with 0(1D). These newer findings suggests that reactions with 0(1D) in the stratosphere would not play a significant role in the degradation of perfluoroalkanes. Ko et al. predict the photo- and oxidative-degradation rates of the perfluorochemical based on UV absorption spectra and assumed quantum yields. They conclude that photodegradation would not occur in the troposphere. Calloway et al. further evaluate the UV absorption spectra of perfluoroalkanes and perfluoro-aromatic molecules. This work shows that absorption spectra of perfluorocarbons occur at wave lengths too short to allow direct photodissociation in the troposphere. UV absorption maxima of perfluoroalkanes are generally below 190 nm. After intense study and with performing key independent experiments, the evidence is clear, highly fluorinated fluorocarbons are an ideal medium in storing/suspending electronically modified oxygen derivatives (EOMDs) and benzo-γ-pyrone reaction intermediates, that otherwise could not be suspended in any other medium, this is due to the extreme reliance's to oxidation, in essence PFCs cannot be further oxidized, because they are already are fully oxidized. One aspect of this invention discloses a method that directly solves the main draw backs in ozone therapy, were ozone gas can be directly dissolved within a fluorocarbon matrix to be used for therapeutic purposes, another aspect of the invention is where I use ozone or oxygen in a fluorocarbon to drive reactions in solution which oxidizes and activates compounds such as benzo-γ-pyrone derivatives, and yet another embodiment of the invention is where I use a fluorocarbon as a dielectric to precisely control reactions when a applied voltage is applied, where the fluorocarbon is used as an dielectric medium to oxidize bio active compounds. When bio active compounds such as benzo-γ-pyrone derivatives are activated within a PFC solution and electronically modified, upon contact with a substrate, the benzo-γ-pyrone compound immediately reacts with the biological substrate, thus bypassing key biological and chemical pathways. This invention solves numerous problems, not only is it relevant for current ozone administering techniques, but your able to drive reactions in solution, that change the nature of compounds, reactions involving intermediates, we can perform and stabilize reactions intermediates that mediate and stimulate cellular responses, from apoptosis to regeneration of tissues.

Oxygen free radical therapy and its derivatives have been used by medical practitioners for the last 50 years in most parts of the world. EMOD effects have been studied extensively for the better part of the last 100 years, with ozone therapy being the most popular form of electronically modified oxygen derivatives. Over the last 25 years there has been a steady increase of clinics that offer EMODs as a therapy in the U.S, but many more in Europe, for instance Germany has over 7000 medical doctors trained in offering ozone therapy, the use of EMOD therapy has been fully adopted in most parts of the world including Europe, south America, Russia, India, Cuba, Spain, uk with an increasing number of clinics opening up every year in the U.S., the truth is, the therapy has sever draw backs discouraging wide spread use in the U.S.. This invention solves the draw backs inherit in modern ozone therapy, and introduces techniques for stabilization of ozone and other reaction intermediates, for therapeutic uses.

EMOD's or Electronically modified oxygen derivatives are oxygen derivatives that are created though reduction-oxidation reactions, commonly referred to redox reaction, some species include 0̂3,0̂-1,0̂-2,0̂4. The unpaired electrons of oxygen readily reacts to form other partially reduced highly reactive species in vivo, including hydrogen peroxide (H₂O₂), hydroxyl radical, and peroxynitrite. Ozone is one form of an electronically modified derivative that is a bluish colored gas and has a boiling point of −112° C. Ozone Can only partially dissolve in water and much more soluble in inert non polar solvents such as fluorocarbons. At −112° C., ozone condenses to form a dark blue liquid at (STP), the solubility of ozone is thirteen times that of oxygen in a aqueous medium. The oxidation potential of 2.07 volt proves that ozone is a strong oxidizer. Ozone is fairly unstable in a watery solution; its half-life in water is about 20 minutes. In air, ozone has a half-life of 12 hours, which makes the stability of ozone in air superior and at temps of −50 degrees C. ozone can be stable for about 3 months in air, Ozone is diamagnetic, which means that its electrons are all paired. In contrast, O2 is paramagnetic, containing two unpaired electrons. As oxygen absorbs electrons and is electrically modified, it forms clusters of O3 and even higher forms of O4, O5 and Ô 6. In the presence of water, ozone breaks down to O2 plus Ô-1. In the process of breaking down, ozone releases electrons into the water. The difference between hydrogen peroxide and ozone is electrons. While both are oxidizers, only ozone releases free electrons. Because of this unique quality, ozone can destroy and reacts with other free radicals such as hydroxide radicals.

### Phsyilogical Effects of Ozone

The physiological effects of ozone is well documented over the years, in 1940, Kleinmann showed the effects of ozone in killing bacteria, properties of ozone which is used today to treat water. Fish observed when ozone is used topically there was a therapeutic effect for various skin diseases. Wolff in 1974 described a method in were a quantity of blood being exposed to ozone and then re-induced into a patient was documented as therapeutic in nature. Since then ozone has been used in therapy with often surprising therapeutic results. Recently the medical community has begun to show serious interest in the topic, despite the fact that thousands of doctors throughout the world have been using ozone in various applications with positive results. The main therapeutic use of ozone today is called ozone autohemotherapy (OAHT) which was documented by Wolf. Recent studies on the mechanism of action have shown that contact between ozone and blood gives rise to effects that can be exploited in medicine. Exposure of human blood to a mixture of oxygen and ozone is not toxic, providing exposure times and concentrations are appropriate. Unlike the respiratory system, human blood, which is in a dynamic state, is able to neutralize the oxidizing power of ozone by a potent defense system. Like other gases (O2, CO2,), ozone must be dissolved in aqueous solution in order to act at the biochemical level. On contact with blood, ozone dissolves in plasma and instantly decomposes as a cascade, for example (h202) hydrogen peroxide, superoxide anion (O2.⁻) and hydroxyl radical (OH.). These compounds are highly reactive with a short half-life. EMODs are produced naturally by the body during cell respiration in mitochondria and during bacterial phagocytosis by leucocytes under times of stress and infection. Humans defend themselves from continuous invasion from pathogenic agents by the production of hydrogen peroxide and hypochlorite radicals. EMODs have their own toxicity, however aerobic organisms have developed an potent antioxidant system, consisting of substances in blood plasma, such as uric acid, ascorbic acid, albumin, vitamin E, bilirubin, intracellular enzymes such as superoxide dismutase (SOD), catalase (T), transferase (GSH T), glutathione peroxidase (GSH-Px), glutathione reductase (GSH R), glutathione and the redox system of glutathione (GSHGSSG), these antioxidants are kept at optimal level by enzymes and the pentose cycle via NADPH. Most of the dose of ozone that comes into contact with blood is partly reduced by hydro soluble antioxidants and partly transformed into EMODs and Lipid peroxide products (LOPS), which are checked by the antioxidant system before they can damage healthy blood cells and tissues. Pharmacological effect of ozone is due to the slight excess of EMODs acting as chemical messengers on membrane receptors, while LOPS act on practically all cells after a blood reinfusion of ozone. The oxidizing action of ozone leads to the formation of hydrogen peroxide that enters cells with various effects; in red blood cells ozone shifts the hemoglobin dissociation curve to the right and facilitates release of oxygen, in leucocytes and endothelial cells induces production of interleukins, interferon, trans forming growth hormone (TGF), and nitrogen oxide, in platelets ozone induces release of growth factors cells, it stimulates long term efficiency of antioxidant systems in adaptation to its oxidizing action. On contact with blood, ozone causes a transitory imbalance between oxidants and antioxidants, as an acute, exogenous oxidative stress. With appropriate exposure time and ozone dose, the oxidative stress may be exactly calculated and transient with respect to endogenous toxicity of EMOD produced over a lifetime. This calculated imbalance activates messengers that trigger biological effects, without exceeding the capacity of the antioxidant system. Ozone, therefore, acts like a drug with a precise therapeutic window. Another effect, that needs further study is a chemotaxes effect, were ozone effects attracts and stimulates activation of endogenous stem cells. Ozone is not toxic if administered within the therapeutic range, but it may be ineffective if the dose is too low, and will be totally quenched by antioxidants. A further aspect of its action could be important and is currently being researched. It regards the capacity to positively regulate the antioxidant system. The body is bombarded by continuous production of EMOD. For example, production of EMODs is high during respiration, in the metabolic cycle of fatty acids, in cytochrome P450 reactions to xenobiotics, in the presence of phagocytosis and in many pathological situations. There are situations over of a lifetime in which a vicious circle of imbalance between production and neutralization of electronically modified oxygen derivatives develops; EMODs continue to increase while the antioxidant system becomes weaker. This happens during chronic viral infections, atherosclerosis, tumor growth, neurodegenerative diseases and aging. Excessive production of EMOD may become chronic and irreversible at certain times, leading to death. Administration of exogenous antioxidants could, at best, slow down the process, but if the latter is not too advanced, prolonged ozone therapy with therapeutic and progressively increasing doses, may restore the balance between EMODs produced and neutralized, this stimulates the antioxidant system, which can adapt to chronic oxidative stress. We know that cells may react to oxidative stress in two ways, if the stress is excessive and continuous, the cell dies; if the stress is modest and transient, the cell has time to react and become resistant, activating expression of silent or rarely expressed genes and producing shock proteins, such as heat shock protein (HSP), glucose-regulated protein (GRP) and oxidative shock protein (OSP). Production of all these proteins is stimulated during ozone therapy. Ozone activates the enzymes involved in peroxide or oxygen “free radical” destruction i.e. glutathione, catalase, sod accelerates glycolysis functioning of red blood cell metabolism. Ozone Increases leukocytosis the production of the white blood cells and phagocytosis (the manner in which certain white blood cells destroy foreign matter). Both processes are part of the immune defense system. Ozone stimulates the reticulo-endothelial system, the rebuilding of tissue. Ozone is Strong germicide—inactivates entero viruses, coliform bacteria, saphylococcus aureus and aeromona hydrophilia. Ozone disrupts the cell envelope of many pathogenic organisms which are composed of phospholipids, peptidoglycans and polysaccharides. Ozone opens the circular plasmid DNA which lessens bacterial proliferation. Low doses of ozone stimulate the immune system. High doses inhibit the immune system. (Breakdown of glycogen) in RGSs, Ozone Enhances formation of acetyl coenzyme-a, which is vital in metabolic detoxification. Ozone Influences the mitochondrial transport system which enhances the metabolism of all cells and safeguards against mutagenic changes. Ozone Increases red blood cell pliability, blood fluidity and arterial P02 (oxygen content) and a decrease clumping of blood. Ozone is neutralized by healthy cells, by the antioxidant system in each cell, damage cells, viruses, bacteria, do not have these antioxidant system or damage cells can no longer catalyze free radicals.

### Poperties of Flurocarbons

PFC liquids dissolve large volumes of oxygen. PFCs are linear, cyclic or polycyclic hydrocarbons in which hydrogen atoms have been substituted with fluorine. The two compounds most widely used in biological systems are perfluorodecalin (C10F18), a bicyclic per fluorinated alkane, and the preferred fluorocarbon of this invention. The other one is bromoperfluoro-n-octane (empirical formula: C8F17Br, known by the generic name of perflubron), a linear molecule with a terminal bromine atom. Liquid PFCs are colorless, odorless and have specific gravities about twice that of water. PFCs were first produced commercially during World War II as part of the Manhattan Project, in the search for inert handling materials that could resist corrosion by the highly reactive uranium isotopes being synthesized for the first atomic bomb. PFCs are extremely inert owing to the high strength of the carbon-fluorine bond (480 kJ mol−1) and the protective effects that the large, electron-rich fluorine atoms lying on the underlying carbon backbone, shielding it from chemical or enzymatic attack. The higher the flouring count the stronger the bonds become, and the more shielding against oxidizing agents like ozone, and reactive carbon oxy intermediates, typically it takes extreme temps above 400 c to see any type of degradation in highly fluorinated fluorocarbons. The standard oxidation-reduction potentials do not apply to most PFCs. The materials are unaffected by electrochemical reactions and do not dissociate in aqueous media. They are essentially already fully oxidized and are unaffected by standard oxidizing agents such as permanganates, chromates, etc. The only known oxidation takes place only at high temperatures by thermal decomposition. Likewise, the materials are only reduced under extreme conditions, requiring reducing agents such as elemental sodium. Commercial applications of PFCs include their use as industrial lubricants, Lasers, coolants and anti-corrosion agents. Teflon or poly(tetrafluoroethylene), the solid protective anti-stick coating on household cookware and frying pans, is a polymerized and highly corrosion resistant PFC. The inertness of PFCs also make them uncreative in the body. The molecules are sequestered by phagocytes cells of the monocyte/macrophage lineage (Formerly known as the reticuloendothelial system). They subsequently diffuse back into the blood where they are carried in plasma lipids to the lungs and exhaled intact as a vapor. Gas solubility of PFCs has the highest gas-dissolving capacities of any liquids. The solubility of respiratory gases, for example, is related to the molecular volume of the dissolving gas and decreases in the order CO2 O2>N2. The solubility of oxygen in PFC liquids (37° C., 1 atm) used for biomedical applications is 40-50 vol. %, as compared to 2.5 vol. % for water; carbon dioxide solubility in the same liquids can be >200 vol. %.Unlike the active binding of oxygen to the hem sites of Hb, oxygen dissolution in PFCs is a passive process, in which gas molecules occupy cavities within the PFC liquid. Consequently, in contrast with the sigmoid binding curve of oxygen to Hb, the solubility of the gas in a PFC liquid at a given temperature is directly proportional to the pO2, essentially obeying Henry's Law Of all the perfluorocarbons, perfluorodecalin has probably seen the most interest in medical applications. Most applications utilize its ability to dissolve large amounts of oxygen 100 ml of perfluorodecalin at 25° C. will dissolve 49 ml of oxygen at (STP) and ozone will dissolve 13 times more than oxygen at (stp). Perfluorodecalin was one of the many ingredients in Fluosol, an artificial blood product developed by Green Cross Corporation in the 1980s. It is also being studied for use in liquid breathing. For a fluorocarbon to be used intravenously, an emulsion must be created, fluorocarbon particles are coated with an adherent lipid which will not be rejected by the recipient at the same time used as the emulsion agent, lecithin is commonly used as a surfactant reactant, Similarly a variety of surfactants reactants can be used, including fluorinated surfactants may be used to form emulsions in accordance with the present invention. Like additives in the aqueous phase, surfactants are chosen according to the desired properties of the emulsion. Examples of suitable surfactants for use in the present invention include lecithins, polyoxyethylene-polyoxypropylene copolymers, sorbitan polyoxy-ethylenes, and phospholipids such as egg-yolk, soy or synthetic lipids, perfluoroalkyl phospholipids and the other synthetic perfluoroalkyl surfactants. Emulsification is achieved usually by ultrasonic vibration (sonication), other methods of Manufacturing are high-pressure homogenization.

### EMODs and Cancer

In cancer the relevance of oxygen and its derivatives for cancer are significant. Specific biological pathways are urgently needed for the development of rationally targeted therapeutics. Electronically modified oxygen derivatives and their role in cancer cell response to growth factor signaling and hypoxia are emerging as areas of exploration on the road to discovering cancer's weakness. Dr. Warburg the most prominent cancer researcher of the 20^{th} century was fist to observe if you lower oxygen 35% on normal healthy cells, in a few days healthy cells will turn cancerous, and he showed the rate of glycolysis can vary over 100-fold over a normal cell in some instances. All cancer cells exhibit hypoxia with an increase in the glucose metabolism and is the hall mark of all cancer cells, all cancer cells oxidize glucose for atp energy production and the dramatic increase in glucose leads to more than normal EMOD production, In malignant tumor cells, the antioxidant systems are elevated in cancer cells to balance the high level of oxidant species being produced when normal respiration is disrupted. The elevation depletes the antioxidative capacity in tumor cells; we can take advantage of over taxed antioxidant system in tumors, by introducing more EMODs, were healthy cells can neutralize the newly introduced EMODs, while cancer cells with their depleted antioxidant system can be pushed over the edge, the present invention introduces methods for creating and or delivering EMODs and EMOD precursors to lead to redox signaling-mediated apoptosis in cancer.

There has also been new experimental evidence performed at Boston medical to support and explain the Warburg effect. Experimental evidence indicates that the key phospholipids responsible for program cell death are being inhibited from releasing Cytochrome C (Cyt C) into the cytosol, the phospholipids responsible is cardiolipin CL. This new evidence may bring light to cancer morphology. Cytocrhome C inhibition seems to be one the mechanisms responsible for the reason cancer cells divides uncontrollably, and may be the reason check points fail in the cell cycle, if a damaged cell cannot start the apoptosis program, its destined to grow and divided uncontrollably.

One of the distinguishing and near-universal hallmarks of all cancers is hypoxia and increase uptake of glucose. Unregulated cellular proliferation leads to formation of cellular masses that extends beyond the resting vasculature, resulting in oxygen and nutrient deprivation. The resulting hypoxia triggers a number of critical adaptations that enable cancer cell survival, including apoptosis suppression, altered glucose metabolism, and an angiogenic phenotype. Recent investigations suggest that oxygen depletion stimulates mitochondria to elaborate increased EMODs, the cell is trying to commit suicide, but with subsequent activation of signaling pathways, such as hypoxia inducible factor 1α, that promote cancer cell survival and tumor growth. Because mitochondria are key organelles involved in chemotherapy-induced apoptosis induction, the relationship between mitochondria, EMOD signaling, and activation of survival pathways under hypoxic conditions has been the subject of increased study. In this present invention we describe mechanisms involved in EMOD signaling and may offer novel avenues to facilitate EMOD-mediated signaling in cancer cells and its potential as a target for developmental therapeutics.

In apoptosis mitochondrial reactive oxygen species production produces oxidative signaling, O2 in the mitochondria are electronically modified by accepting an electron which lead to the creation of the superoxide anion, which in turn is reduces to h202 and peroxynite. Interactions of cytochrome c (Cyt c) with the mitochondria specific phospholipid cardiolipin (CL) result in a high affinity cytochrome c-CL complex that acts as a specific and potent oxidant. In the presence of hydrogen peroxide, this complex functions as a CL-specific oxygenase catalyzing oxidation of CL. Binding with CL turns off cytochrome c's function as an electron carrier but turns on its peroxidase activity. Oxidized CL has a markedly lower affinity for cytochrome c and abandons the complex. CL oxidation products (CLox; mostly cardiolipin hydroperoxides) accumulate in the mitochondria, leading to the release of pro-apoptotic factors into the cytosol. AIF, apoptosis inducing factor; ANT, adenine nucleotide translocase; VDAC, voltage-dependent anion-selective channel.

Transformed cancer cells commonly lack cell cycle checkpoints and over express oncogene growth factors and tyrosine kinase receptors that drive cell proliferation, ultimately leading to tumor formation and chronic hypoxia. Which leads me to and enzyme that is commonly over expressed in cancer, this enzyme is called Thioredoxin reductase. Thioredoxin reductase is a ubiquitous flavoenzyme from archea to human and the only enzyme able to catalyze the reduction of Trx by NADPH. Mammalian TrxR contains a conserved COOH-terminal active site sequence -Gly-Cys-Sec-Gly together with an NH₂-terminal redox active disulfide.(TrxR) has a broad spectrum of substrates, ranging from small molecules such as selenite, lipid hydroperoxides, ebselen, and dehydroascorbate to proteins like protein disulfide isomerase or glutathione peroxidase. Most of these substrates are involved in cellular redox regulation; therefore, (TrxR) plays a central role in maintaining the redox homeostasis directly or with (Trx) as well. (TrxR) and (Trx) have been reported to be over expressed in many aggressive tumor cells in which the proliferation is crucially dependent on a constant deoxyribonucleotide supply. Accordingly, the inhibition of thioredoxin system can induce cell death or increase the tumor cell sensitivity to other cancer therapies. The thioredoxin system, composed of thioredoxin reductase (TrxR), thioredoxin (Trx), and NADPH, exerts a range of activities in cellular redox control, antioxidant function, cell viability, and proliferation. Recently, the selenocysteine (Sec)-containing mammalian TrxR has become a new target for anticancer drugs.

TrxR and Trx are overexpressed in many tumors and tumor cells seem to be more dependent on Trx system than normal cells. Reaseach has shown (Cancer Res Apr. 15, 2006 66; 4410), that 3-hydroxyl-containing flavonoids such as quercetin, myricetin, taxifolin, catechin, and pelargonidin exhibited an NADPH-, concentration-, and time-dependent inhibitory effect. Flavonoids represent a large family of polyphenolic compounds synthesized by plants. The feature they have in common is their chemical structure, characterized by one or more condensed aromatic rings. Due to such structure, flavonoids have specific color, smell and taste. They exert a wide range of biological activities in addition to their antioxidant activity, which is one of the most important features of their functioning; flavonoids can modulate the activity of enzymes or cell receptors, and interfere with the essential biochemical pathways, suggesting their involvement in biochemical and physiological processes in humans as well in plants. Flavonoids, which are benzo-γ-pyrone derivatives with A, B, and C rings, are categorized as flavanones, flavones, flavonols, anthocyanidins, isoflavones, and flavonols, they show inhibitory effects on thioredoxin reductase, thioredoxin reductase is a key mediator in the cellular response to oxidative stress that is frequently over expressed in cancer. This over expressions is one of the reason of EMOD in the defective cancer cell do not start the apoptosis because they are catalyzed before they can build up to a sufficient level to oxides CL for it to release (Cyt c) into the cytosol to induce cell death. Among the many Flavonoids type compounds that inhibit (TrxR), myricetin and quercetin differ from all other compounds, because these two flavonols are readily auto-oxidizable, these compound are EMOD precursors and readily form the superoxide radical inside the cell. Research has shown flavonols myricetin and quercetin and their oxidized products are both inhibitors and substrates. The interactions of flavonols with TrxR may occur in several steps, as suggested from experimental evidence that is sited.

“Step 1, flavonols inhibit TrxR directly and produces modified TrxR, triggering the inactivation of TrxR. Step 2, modified TrxR produces oxygen radicals or EMODs within the cell via naph. Step 3, the oxygen radicals attack the flavonols to yield O-semiquinone or via auto-oxidization. Step 4, O-semiquinone reacts with active TrxR and inhibits it. Step 5, o-semiquinone can be oxidized further to be quinone methide, an electrophile which can form a conjugate with protein thiols. Step 6, the oxidization of step 5 can be prevented by the active TrxR, which can be inactivated by quinone methide in reverse. Superoxide dismutase or incubation under anaerobic conditions will attenuate superoxide production and diminish the step 4 reaction, whereas xanthine/xanthine oxidase system produces more superoxide and accelerates the reaction of this step. The semiquinone or quinone methide may attack the selenocysteine in COOH terminus of reduced TrxR to modify TrxR and prevent the enzyme from reduction of Trx. Consequently, reduced Trx, which is normally present in the cells as a result of TrxR activity, will be replaced by the oxidized form, which may induce Trx-mediated cell death. So what this means is reduced Trx can bind and inactivate apoptosis signal-regulating kinase 1 whereas oxidization of Trx results in the activation of apoptosis signal-regulating kinase 1 and induction of apoptosis signal-regulating kinase 1-dependent apoptosis.”

Reseach has shown the oxidized version of flavoniods, the semiquinone or quinone methide may attack the selenocysteine in COOH terminus of reduced TrxR to modify TrxR and prevent the enzyme from reduction of Trx. It is also relevant the oxygen content of the substrate is directly proportional to the amount of h202 the cell produces, which means the more oxygen present the greater h202 production within the cell that can act directly on Cyt c for its release into the cytosol, the mere act of creating an oxygenated fluorocarbon emulsion will activate and oxidize flavonoids, Via auto oxidation. Experiential evidence published in (Molecules, 2007, 12, 654-672), describes auto oxidation mechanism, the mere act of bubbling air though a water or a water/ethanol quercetin suspension, oxidized quercetin, and change the molecular nature, what this experiment showed was you can oxidize benzo-γ-pyrone flanonoids, in a mild solution of PH-7, with just oxygen. It should be noted this type of oxidation led to direct cleavage of the C2-C3 bond in the diketo-tautomer of quercetin, changing the skeleton structure, and the author noted that this might be the reason we cannot detect benzo derivatives in the blood, because of similar oxidation events, because the nature of the compound is change, along with chemical signature which has change also. It should be noted bubbling ozone through a PFC solution will steal the hydrogen atom of the benzo-γ-pyrone derivative, the extra 0̂-1 in 0̂3 will steal the hydrogen atom from the skeleton structure directly and lock it up, to produce a o-semquinone.

Perfluorocarbon have high dielectric strengths and high insulating properties, and so can be used in direct contact with high voltage components, either as dielectric fluids, dielectric gases, or as coolants. Which leads me to another method of precisely stealing the hydrogen molecule of a benzo-γ-pyrone derivatives and oxidize it, is where a highly fluorinated fluorocarbon is used as a dielectric with an applied voltage, you start with a flavonoid benzo-γ-pyrone derivates with A, B, C rings then you dissolved in a organic solvent such as ethanol and where a super critical anti solvent is used to precipitate nano-crystalline particles that is vacuum dried and stored prior to emulsification. Disperse the flavonoid crystalline compound throughout the PFC solution with ultra sonic cavitations, and add an electric potential, this will de-protonize the benzo-γ-pyrone derivative, this method can be quite precise, where the auto oxidation cleves the skeletal structure, this method you can rip the hydrogen atoms off and create a highly potent oxidized reaction intermediate without skeletal cleavage, this molecule will directly act on TrxR enzyme and eliminate the biological pathways described earlier to start the apoptosis program immediately on contact with in the mitochondria. This method of oxidation is called, Electrochemical Oxidation, and this method has been known in the art for some time, using water/ethanol solutions, or a silver chloride solution, but in using a fluorocarbon instead of other aqueous solutions, lets you get rid of unwanted reactions, we can also store the oxidized products in a frozen state, for use later in a therapeutic setting. Presently there was a patent filed for COMPOSITIONS AND METHODS OF “CONTROLLING AND ADMINISTERING REDOX SPECIFIC FORMS OF DRUGS, FOODS AND DIETARY SUPPLEMENTS” issued Jan. 22, 2009, the authors are Steven Baugh and Thomas Hnat. This method has been known in the art for some time, and an example of this is in a paper published in 03, “Electrochemical Oxidation of Quercetin”, but there has been no published results to date known that ever used a fluorocarbon as the dialectic material. In the invention published the author uses a battery with a constant applied voltage to a solution at the times before delivery to a patient, but if you use a PFC as the dielectric you can use cryogenic methods to lock the reaction intermediates within the PFC matrix to be used at a later date. So fluorocarbons when used as a dielectric with an applied voltage will de-protonizes the skeletal structure of benzo-γ-pyrone derivative and achieve oxidized forms of these chemical intermediates that can act directly on the TrxR enzyme to induce the apoptosis.

We can now directly deactivate (TrxR) enzyme without going through the 3 initial biological steps your body has to go through to produce the same result; by passing key pathways will lead to an increased efficiency of (TrxR) deactivation. Ironically and paradoxically flavonoids were considered powerful antioxidants and there protective action that you see from flavonoids are from the anti oxidant ability, were the converse here is true, were using the proxidant ability of flavonoids to release the super oxide anion, to induce the cell death program in a cancer cell. Due to the extreme inertness of fluorocarbons, they pose an ideal medium to oxidize and stabilize these reaction intermediates in a frozen or cryogenic state, for study or delivery to the mammalian patient on a later date. The inhibition of thioredoxin system can induce cell death or increase the tumor cell sensitivity to other cancer therapies or molecules introduced into the cocktail. Another aspect of this invention addresses the high levels of glucose cancer cells use to produce ATP, that can be addressed with another EMOD precursor and osmotic agent for the invention to maintain blood osmolarity is 2-Deoxy-D-glucose, 2DG is a glucose molecule which has the 2-hydroxyl group replaced by hydrogen, so that it cannot undergo further glycolysis. Many cancers have elevated glucose uptake and hexokinase levels. 2-Deoxyglucose labeled with tritium or carbon-14 has been a popular ligand for laboratory research in animal models, where distribution is assessed by tissue-slicing followed by autoradiography, sometimes in tandem with either conventional or electron microscopy. 2-DG is up taken by the glucose transporters of the cell. Therefore, cells with higher glucose uptake, for example tumor cells, have also a higher uptake of 2-DG. 2-DG showed that in cancer cell cultures that it hampers cell growth, and induces apoptosis by glucose deprivation. Another chemical and anti glycolic agent used as a buffer in my invention, to maintain osmolarty are salts dichloroacetic acid with potential antineoplastic activity. Dichloroacetate ion inhibits pyruvate dehydrogenase kinase, resulting in the inhibition of glycolysis and a decrease in lactate production. This agent may stimulate apoptosis in cancer cells by restoring normal mitochondrial-induced apoptotic signaling. With this unique combinations of glucose inhibition, high oxygen caring capabilities of fluorocarbons, and TrxR enzyme inhibitor, pose a potent therapy against many cancers, that is relatively non toxic to normal cell healthy cells, because we're targeting cancer cells directly and taken advantage of the over taxed antioxidant system, and depriving cancer of glucose.

### EMODs and Tissue Regeneration

EMODs appear to be gaining an increasingly important role in the modulation of cellular proliferation and cellular death. EMODs offer a therapeutic site in the selective killing of neoplastic damage cells, without causing harm to normal cells. The potential of therapeutically delivering EMODs and or its reaction intermediates in an inert bio-compatible solution offers a new powerful way to treat many diseases and will play an important role in wound management also. For instance the closure of cutaneous wounds, wounds commonly seen in diabetic ulcers and burns, involves complex tissue movements such as hemorrhage, inflammation, re-epithelization, granulation tissue formation, and the late remodeling phase of repair. These events involve coordination of dozens of types of cells and matrix proteins, which are all important to control stages of the repair process. Previous studies have demonstrated that endogenous growth factors, such as fibroblast growth factors (FGF), platelet derived growth factors (PDGF), transforming growth factor-β (TGF-β) and vascular endothelial growth factors (VEGF) are the important regulatory polypeptides for coordinating the healing process. They are released from macrophages, fibroblasts, and keratinocytes at the site of injury and they participate in the regulation of re-epithelization, granulation tissue formation, collagen synthesis and neovascularization. It is shown that exposure to EMOD is associated with activation of transcription factor NF-κB; this is important to regulate inflammatory responses and eventually the entire process of wound healing. Many examples show that large amounts of PDGF and TGF-β1 are released from platelets in the heparinized plasma, after EMOD therapy. It has been shown in many experiments that there are substantial increases of steady-state mRNA levels of TGF-β1 in the fibroblasts that were co-cultured with bronchoepithelial cells after Ô-1 exposure. EOMD therapy facilitates acute cutaneous wound healing, and this is associated with growth factors such as FGF, PDGF, TGF-β and VEGF. The unique ability of oxygen Free radicals to induce immune related messenger molecules called cyokines comes from its action on the membranes of white cells. Examples of cytokines are gamma interferon, interleukin-2, colony stimulating factor, and TNF-alpha just to name a few. The use of a pure PFC with ozone can be used to speed the healing process on diabetic ulcers, used as an enema for intestinal ulcers, an ozonated suspension can be used as medium or a delivery method for synthetic extra cellular membranes, an ozonated PFC suspension will stimulate the immune response and tag the damage cells for replacement, were as the ecm is used as a scaffolding material, were new cells can attach and grow; similarly the fluorocarbon in this invention can be used to transport carbon or non reactive gold nano particles or nano particulates for targeted delivery to specific sites, such as a tumor, as it is known PFC's tend to accumulates in the specific tissues, such as tumors.

This invention was first conceived and will be used for ligament injections, called prolo-therapy; prolo-therapy is a form of non-surgical ligament reconstruction and is a permanent treatment for chronic pain. Prolo-therapy is a connective tissue injection therapy with (EMODs), which can reconstruct damaged or weakened connective tissue in and around joints. EMODs are injected into the damaged connective tissue in and around a joint to rebuild the damaged areas. Ligaments are the structural “rubber bands” that hold bones to bones in joints—acting like the body's shock absorbers. Ligaments can become weak or injured and may not heal back to their original strength or endurance. Ligaments will also not tighten on their own to their original length once injured. This is largely because the blood/oxygen supply to ligaments is limited, and therefore healing is slow and not always complete. To further complicate this, ligaments also have many nerve endings, and therefore the person will feel pain at the areas where the ligaments are damaged or loose. Loose ligaments lead to pain in the joints, and if not fixed will always lead to some form of arthritis. Prolo-EOMD therapy is an injection technique that heals joint's much more quickly than other techniques. Growth factors and fibroblasts discussed tighten ligament which gives the joints the ability to fully repair. By stabilizing ozone in a bio compatible PFC is monumental breakthrough for this particular therapy, it solves the solubility problem and the stability problem, prolo-therapy practitioners no longer need to have ozone machines on site, also many animals such as race horses that would have to be put down otherwise because they have chronic ligament and tendon injuries will benefit from this invention immensely. This therapy has been used around the world for the last 50 years, and it's been well documented.

Presently there are only 3 ways in delivering ozone directly to a patient. Ozone gas can be directly injected, dissolved in aqueous solutions such as saline, or dissolved in ones blood for reinfusion. The problems with present methods include the following:
1. Poor solubility in polar fluids, polar fluids cannot dissolve enough of the ozone gas. Because you cannot achieve a therapeutic concentration, some practitioners directly inject ozone gas though IV infusion, which can be extremely dangerous.
2. Short half-life in all mediums used, less than 20 min in saline, by the time you bubble ozone in an aqueous medium and time it takes to administer you lost half of the gas, compounded by the fact most aqueous solutions such as saline have very poor solubility.
3. Ozone must be generated and delivered to the solute on site always.
4. No real way to store highly unstable EMODs, even for short periods of time, with present methods.

There are clinics that use direct Intravenous injections of ozone gas. Ozone gas that is introduced in the vein, this is called I.V. infusions; this method used by some physicians, infused ozone requires continuous monitoring to prevent too much ozone gas from entering the blood at one time; this method could cause an embolism. Small amounts of ozone gas are directly fed into a vein over a period of time, can cause a hardening of the vein at the site of entry, Direct infusion can be dangerous, and is frowned on by most physicians, other parameters must be monitored also, such as concentration, flow rates, and quality of ozone production. The other method and the most popular and Successful method for administrating ozone describe in such as in U.S. Pat. No. 6,569,467, this type of method is called autohemotherpy, The inventor Discloses an autoimmune vaccine were the patient blood is exposed to ozone then re-infused in the patient along the line of Wolff's work. This method has an excellent track record for safety, by far the best method so far for administrating oxygen derivatives to a patient, as described earlier this method uses your blood as the medium of transport, a few obvious problems with this method is blood does coagulates when exposed to the air, though the use of sodium heparin is wieldy employed, an anti coagulant, it has been shown to cause problems in patients with liver problems, besides the heparin issue, you're constantly exposed to patients blood, by using a fluorocarbon suspension, you dramatically limit you exposure and the probability of cross contamination; it's just a cleaner more sound delivery mechanism than using a patient blood for delivery, there are things you can do with a synthetic that cannot be done with a patient's blood, like have bioactive agent or particulates in the suspension depending on the disorder you are treating, by using a synthetic delivery mechanism, you open up the possibility for other uses, such as creams, gels, site injections Intramuscularly, subcutaneously, and intracavitary uses, not to mention we can oxidize and activate other compounds such as flavonoids describe earlier.

### EMODs Concentrations

The volume of ozone per hour, which is what many industrial ozone units are gauged by, is not directly relevant to the therapeutic value's of ozone. The greatest factor to consider is the actual concentration of ozone that a machine produces. 3% Ozone (42 ug/ml in pure oxygen) is the minimum therapeutic concentration, with 5% ozone (70 ug/ml in pure oxygen) being the generally recognized maximum effective concentration. When it comes to concentrations of ozone, this is dependent on the type of ozone machine used, many ozone machines have come alone over the past 100 years of inception, most of them are energy intensive and inefficient, but a new class of machine came along, called a PEM or proton exchange membrane, and is the most efficient thus far in generating ozone, concentration up to 20% w/v to oxygen can be achieved, this threshold can be increased substantially when uv radiation is used in conjunction or simultaneously administrating ozone to the fluorocarbon, high concentration can be achieved if so desired, but more than a 5% ozone concentration in PFC solution will not enhance the immune response when used intravenously, but it may be desirable to have very high concentrations to use less of the solution for purposes of directly injecting in tissues. The source used in a PEM is only distilled water, which can be decomposed into hydrogen, oxygen and ozone, 80% oxygen with 20% ozone mix is exhausted on one side, while hydrogen gas is exhausted by another port. This method is the best up to now, the PEM machine uses 3.5v low-tension DC voltage, which means that the wear on the anodes and cathodes is very low, thus ensuring a long life (the expected life time is longer than 15,000 hours). The other technology used to manufacture Ozone is the air discharge technology which simulates a lightning strike. Air which consists of oxygen (21%), hydrogen (78%) and other gases when subject to certain discharge period, high-tension currents higher than 10,000v are made to make ozone gas, heat produced causes the oxygen to break bonds to form ozone. Some NOX nitrides are produced at the same time, which is internationally known as poisonous and can cause cancer. There is little possibility that high-purity ozone is made with air discharge technology. Because the amount of oxygen in the air is limited and the usage of high-tension Current and wearable electrodes restricts the useful life time and Reduces the safety limits.

### SUMMARY OF INVENTION

This invention discloses 9 main embodiments how to create, store and deliver EMODs and or EMOD producing precursors to a patient.
1. Were a flavonoid benzo-γ-pyrone derivatives with A, B, C rings in this invention are dissolved in a organic solvent and were a super critical anti solvent is used to precipitate nano-crystalline particles that are vacuum dried and stored prior to emulsification.
2. Where a fluorocarbon emulsion is created with, surfactant agents, buffers, osmotic agents, benzo-γ-pyrone prone derivatives, where the emulsion is delivered intravenously to a patient, were ozone/oxygen is delivered simultaneously into a PFC solution at the moment of infusion. Designed to be used in ozone clinics, and veterinary settings, expensive dialysis machines to ozonate the blood becomes obsolete, and this method eliminates the blood removal method.
3. Where a PFC emulsion, emulsified by ultra sonic cavitation, with little or no surfactant, buffer's or additives, beside pure water, were high concentrations of ozone are preferable, where the solution is cryogenically frozen immediately, for direct injection into tumors, to induce apoptosis through necrotic death.
4. Where a pure fluorocarbon continuous phase with only dissolved oxygen and ozone, For the purpose of injection, topical use, burns, ulcers, diabetic ulcers, tendons, ligaments and or intracavitary, for intestinal ulcers. The ozone can be cryogenic frozen or just a normal frozen state, it might be advantageous to include synthetic extra cellular type membrane material that resist oxidation within the PFC solution to induce cartilage regeneration. Or it might be advantageous to include benzo-γ-pyrone derivatives also for direct injection inside cancerous tumor cells.
5. An oxygenated PFC emulsions, with proper buffers, osmotic agents, suspended with benzo-γ-pyrone flavonoid derivatives, glucose derivatives (2dg), were oxygen within the solution auto-oxidizes benzo-γ-pyrone derivatives, to be used intravenously. The oxygen attacks the flavonols via auto-oxidization, which reacts with active (TrxR) and inhibits it.
6. An oxygenated PFC emulsions, with proper buffers, osmotic agents, suspended with benzo-γ-pyrone flavonoid derivatives, glucose derivatives (2dg), were ozone/oxygen is used to drive reactions to activate benzo-γ-pyrone derivatives, to be used intravenously. Can be stored in a frozen state, the point here is the ozone is used to drive reactions and not necessarily has to be present at the time of infusion, the purpose of ozone is to activate the benzo-γ-pyrone flavonoid derivatives to inhibit TrxR. Method of storage can be a slow freeze, or cryogenically frozen immediately after creation, were the activated flavonoid and ozone, both which are hydrophobic is stable within the micelle structure of the PFC emulsion at low temps of −50 C.
7. Where two PFCs emulsions are created that are physically separated, one with the oxidant such as oxygen or ozone/oxygen and the other having benzo-γ-pyrone derivatives with an A-B-C skeletal structure, were the oxidant comes in contact with the flavonoid at or before infusion in one blood stream, and were the bulk of reaction happens inside the body.
8. Where the fluorocarbon is used as a dielectric with an applied voltage, for example you can accurately rip hydrogen molecules off of a flavonoid skeletal structure to produce benzo-γ-pyrone reactive intermediates, once in active form you can instantly freeze using cryo methods for long term storage for later study or use, or just in normal refrigerated conditions, can be used topically, for burns ulcerations, skin cancer.
9. Where I can choose to use a combination of the methods described.

### DETAILED SUMMARY OF INVENTION

An important aspect of the present invention is choosing and preparing a perfluoro compound suitable for in vivo and ex vivo administration. Perfluorochemical molecules have very different structures that have very different physical properties such as gas solubility, density, viscosity, vapor pressure, and lipid solubility. Thus, it is critical to select the appropriate PFC for a specific biomedical application, because the administration may be intravenous, subcutaneous, intramuscular, topical and intracavitary. Not only one must choose a proper PFC the preparation is equally important. For intravenous use, an emulsion with surfactant reactants, buffers and osmotic agents, must be employed. Emulsions are dispersions of two or more immiscible liquids. When an a emulsion is created through sonification, highly intensive ultrasounic waves supplies the power needed to disperse a liquid phase (dispersed phase) in small droplets in a second phase (continuous phase). In the dispersing zone, imploding cavitation bubbles cause intensive shock waves in the surrounding liquid and result in the formation of liquid jets of high liquid velocity. If a cavitation bubble implodes near the phase boundary of two immiscible liquids the resultant shock wave can provide a very efficient mixing. Stable emulsions produced by sonication are used in the textile, cosmetic, pharmaceutical, food, and petrochemical industry. Ultrasonically generated emulsions are more stable and require less if any surfactant than those produced conventionally. Since ultrasound is fully controllable and adaptable by the choice of amplitude, pressure and temperature, sonification is an effective instrument to obtain emulsions with smaller droplet sizes within a narrow size distribution. Cavitation is the formation of vapor bubbles during the negative pressure cycle of ultrasound waves. The bubbles can collapse, resulting in localized high temperatures and pressures. Free radicals, such as the hydroxyl radical, singlet oxygen, and solvated electrons are typically generated from bubble collapse in aqueous media.

To make perfluorocarbons suitable for IV infusions, the perfluoro particles must have a coating material which masks the surface of the perfluoro chemical, while imitating the outward appearance of normal red blood cells. The PFC medium should also contain the necessary electrolytes or salts in proper concentrations to make the emulsion isotonic with respect to blood plasma. It is preferable use a lipid to coat particles of the PFC. The preferred lipids are phospholipids such as lecithin, the source of lecithin is egg yolk, or soy lecithin, and also many surfactant reactants can be used such as fluorinated surfactant reactants. The aqueous phase generally has an osmolality of approximately 300 mOsm and the osmotic agent may be polyethylene glycol, propylene glycol, hexa-hydric alcohol such as mannitol or sorbitol, or a sugar such as glucose, mannose, 2-deoxy-D-glucose 2DG, 2-Deoxy-2-(18F)fluoro-D-glucose fructose. 2-deoxy-D-glucose 2DG is also the anti glycolic compound. Many buffering agents can be selected such as sodium chloride, sodium bicarbonate, magnesium chloride, mono- or dibasic potassium phosphate, calcium chloride, magnesium sulfate, or mono- or dibasic sodium bicarbonate also imidazole or tris-hydroxymethyl-aminomethane, sodium dichloroacetate, potassium dichloroacetate, and diisoproyl ammonium dichloroacetate, dichloroacetic acid.

Not all perfluoro compounds are suitable in blood preparations as one should expect, though there are a few that are chosen for their exceptional properties. U.S. Pat. No. 4,497,829 referenced above teaches us that Fluorodecalin have been found to be the best, in terms of the speed of elimination from the body, but may not be easily emulsified, fluro compounds tend to accumulate in organs such as the liver and spleen, and other tissues and it has been shown that Fluorodecalin (C10F18) to be the best in that aspect and is eliminated the quickest. Though many others can be chosen for slightly different properties, like ease of emulsification, or can carry slightly more dissolved gas, it might be preferable to choose one that accumulates in tissues such as cancer; it might be advantageous to use ultrasound to cause exploding cavitation with in the cell. The preferred PFC for this invention is Fluorodecalin C10f18, not only can it be eliminated the fastest from the body, but the structure and the fluorine arrangement protect the carbon bond from all oxidation, The electron rich fluorine arrangement creates a force field around the decalin bicyclical structure that cannot be penetrated from negatively charged radicals. The chemical stability of an emulsion is important and reflects its resistance against chemical changes, mostly oxidation of fats, this can be addressed by the use and addition of antioxidants in the emulsion or using surfactant reactants that are synthetic in nature. By using ultrasonic cavitation to create the emulsion, ultra fine particulates is created by this process, we can have a much more stable emulsion which can takes advantage of Vander waal forces between the particulates, though not taught in U.S. Pat. No. 4,497,829, but that is precisely the reason for the enhanced stability. It is possible to eliminate all surfactant emulsification with ultra sonic cavitations.

Preferred concentrations of components for selected emulsions or gels that are compatible with the present invention are generally as follows:

From 10 to 125% in weight/volume of an oily phase,

from 0.1 to 12% in weight/volume of surfactants, and

the aqueous phase and buffers making up the balance.

Micro emulsions may be prepared with the following ratios:

from 10 to 125% in weight/volume of an oily phase,

from 3 to 35% in weight/volume of surfactants, and

the aqueous phase and buffers making up the balance.

Once a suitable emulation is created to spec, with the correct balance of buffers, surfactant reactants, anti glycolic inhibitors, electron chain blockers, flavonoids, with a ph balance between 6.5 to 8. You can store until you're ready to add ozone, if applicable at all. If applicable for treatment, a small amount of Ozone gas can be bubbled through the solution to oxidize and activate benzo-γ-pyrone precursors as described earlier were the activated hydrophobic flavonoid is stable at low temps in the micelle of the solution.

A stable emulation can be created with very little or totally without surfactant reactants and other additives. A stable emulsion up to up to 20% pure water can be achieved, through ultrasonic cavitation ; this type of emulsification process creates ultra fine particulates that employ the uses of weak molecular attraction (van der Waals force) between hydrophilic particles, in this method with can eliminate the emulsifying agent that can be prone to oxidation, this method of emulsification is ideal for direct injection in tumors with high concretion of potent radicals. When you begin to bubble ozone through the emulsion in doing so leads to the creation of the superoxide radical, a mass fraction of which is created with a chain reaction, initiating the reaction is OH ion. The superoxide anion is the key anion in the mitochondria to start the oxidation process for programmed cell death; healthy cells can catalyzed this anion with the enzyme superoxide dimatuse (SOD). Emulsion created with high concentration of ozone would only be used to inject into specific sites such as tumors cells, to induce cell death, depending on the concentration this will be necrotic death.

Initiators of the free-radical reaction are those compounds capable of inducing the formation of superoxide ion O2— from an ozone molecule. These are inorganic compounds (hydroxyl ions OH—, hydroperoxide ions HO2— and some cations), organic compounds include (glyoxylic acid, formic acid, humic substance). Promoters of the free-radical reaction are all organic and inorganic molecules capable of regenerating the O2̂-2 superoxide (which can promote the decomposition of ozone) anion from the hydroxyl radical. Common promoters that are also organics include aryl groups, formic acid, glyoxylic acid, primary alcohols and humic acids. Among the inorganic compounds, phosphate species are worth a special mention. Contrary to those of ozone, OH-radical reactions are largely a-selective.

Indirect reactions in an ozone oxidation process can be very complex. Indirect reaction takes place according to the following steps:
1. Initiation
2. Radical chain-reaction
3. Termination

The first reaction that takes place is accelerated ozone decomposition an type of initiator. This can be an OH-molecule

1: O3+OH—→O2.—+HO2.

This radical has an acid/base equilibrium of pKa=4.8. Above this value, this radical no longer splits, because it forms a superoxide radical, see Reaction 2:

2: HO2.→O2.—+H+(pKa=4.8)

Radical chain-reaction

Now, a radical chain-reaction can takes place, during which HO radicals are formed. The reaction mechanism is as follows:

3: O3+O2.—→O3—.+O2 4: O3.—+H+→HO3. (PH<≈8) 5. HO3→O3.—+H+6. HO3→HO.+O2

The HO radicals that have formed react with ozone according to the following reaction mechanism:

7: HO.+O3→HO4. 8: HO4.→O2+HO2.

During the last reaction, HO2. radicals are formed, which can start the reaction all over again (see reaction 2). The reactions of ozone with aqueous solutions are key for Superoxide ion creation that is responsible for to starting the apoptosis program. Promoters are substances that transform OH-radicals to superoxide radicals. Various substances can become promoters, including organic molecules.

When the aqueous solution is agitated by sonofication or UV light, peroxides are formed. Ozone reactions with hydrogen peroxide splits in water according to the following reaction:

2O3+H2O2→2 OH.+3 O2

And

H2O2→HO2—+H+HO2—+O3→O3.—+HO2. HO2.→O2.—+H+O3.—+H+→HO3 HO3→HO.+O2HO.+O3→HO4.HO4.→O2+HO2.

End results of these reactions produce HO2. radical which can start the reaction over again, that leads to the superoxide radical. The reaction with hydroxyl and hydro peroxide ions can be considered the main initiation reactions of ozone decomposition in water, other initiation agents is hydrogen peroxide, direct photolysis (uv) and sonification of ozone yields hydrogen peroxide and then free radicals. The reaction of ozone and the superoxide radical is one of the main components of the ozone decomposition mechanism. Promoters are those species through their reactions with the hydroxyl radical, propagate the radical chain to yield the key free radical; the superoxide radical.

Hydrogen peroxide is an initiator agent of ozone decomposition, but it can also act as a promoter, through these reactions, that will eventually lead to the superoxide radical.

HO.+H2O2→HO2.+H20

HO.+HO2—.→HO2.+OH

The OH* compounds are radicals that contain a very high electronic potential, which makes it one of the strongest oxidizers known. The activation of OH* radicals is a very complex process, which can take place according to a variety of different reaction mechanisms. These reactions give ozone's its disinfection and sterilization abilities, and the super oxide radical produced in the above reactions is the key to induce apoptosis in a cancer cells. Also free radicals spread rapidly and permeate through the cell wall of bacteria; the strong oxidation effect can denaturalize bacteria's albumen and destroy their enzyme system, leading to its decomposition and which leads to death. This type of Free radical cocktail has the ability to deactivate bacterial Infections; a partial list of organisms susceptible to inactivation Includes both aerobic and anaerobic bacteria: Bacteroides, Campylobacter, Clostridium, Corynebacteria, Escherichia, Klebsiella, Legionella, Mycobacteria, Propriobacteria, Pseudomonas, Salmonella, Shigella, Staphylococcus, Streptococcus, and Yersinia. Indeed, all bacteria, including Mycobacteria known for their robust cell walls Succumb to ozone's killing action.

In this invention we can stimulate Apoptosis by 3 main routes, we can induce apoptosis through the mitochondria (the intrinsic pathway), and the free radicals also stimulate the activation of death receptors (the extrinsic pathway) on the outer membrane. Both pathways converge to induce the activation of caspases the final executioners of cell death, although, it should be noted that are compound also induces caspase-independent forms of apoptosis, when injected directed in a tumor and depending on the concentration of highly reactive free radicals they oxidize organelles including mitochondria, endoplasmic reticulum and lysozymes, leading to an increase in calcium and the release of effector proteins, this is frequently involved in caspase-independent cell death, which is a necrotic death.

Many attempts have been tried to induce apoptosis in the cell through different biological path ways using chemotherapy drugs, but due to the extreme hypoxia seen in all cancers, and over expression Thioredoxin reductase, these attempts fail, there is not enough oxygen present to act as an electron acceptor to start apoptosis process, and whenever any oxygen radials are created in the hypoxic cell, TrxR will quickly neutralize it, in other words oxygen free radicals quickly gets neutralized, and not enough is present to a induce a conformational change in CL, but by injecting stable oxygen radicals, with activated flavonoids in a tumor, you eliminate these path ways, and let the free radicals oxidize CL. It should be noted some fraction of ozone will also pass through the cell membrane then dissociate within the cell to oxidize CL to release Cyt(c) into the cytosol. In this invention we are targeting the chemistry inside the cell that is responsible for releasing pro-apoptotic factors into the cytosol to induce cell death, not just attacking fast growing cells indiscriminately like so many modem chemotherapy drugs, this is truly targeted therapy. The bodies' chemistry is strait forward for apoptosis, now we can stabilize the same intermediates your body produces that start apoptosis program in essence we can control apotosis now with these stable free radicals.

This method of using inert PFC's to deliver EMODs and other electronically modified intermediates are superior to other methods previous invented, such as compared to administrating ozone in a non inert polar fluid, or in ones blood. EMODs and Reaction intermediates disclosed in this invention can be used in conjunction or a stand alone, depending on treatment and preparation.

### Experimental Evidence for Stabilization of Ozone in a PFC Solution

### Experiment 1

15 ml of distilled h20 was used to bubble a mixture of ozone/oxygen through the sample for 30 min in a glass bubbler using a custom made proton exchange membrane ozone machine, which then 0.01 ml sample was quickly placed in a hatch DR 4000 u spectrometer, the machine was zeroed before the sample was placed in, also the absorbance wave length was set for 260 nm, the detection absorbance band of ozone, initial results at the peak were 0.590 ABS, but quickly decayed and after 20 min the ozone present in the sample was completely gone. This is the main problem with modem ozone therapy, the sample disappears with a predictable results extremely fast decay rate with in 20 min, and there is not enough of a concentration left to make a therapeutic difference, especially for burns, ulcerations, tendon injections.

### Experiment 2

15 ml of 95% pure Fluorodecalin (C10F18) was placed in a glass bubbler were 20/80 percent mix by weight of ozone/oxygen was bubbled from a custom PEM ozone machine for 20 min, which then proceeded to take a 0.01 ml sample to analyze on a hatch DR 4000 u spectrometer at a wave length of 260 nm, the absorbance band of ozone, while simultaneously had an assistant immediately place the rest of the sample in the freezer at −50 C. The absorbance over time was taken for two consecutive ten hour runs, were the second run was started immediately after the first, initial reading were around 0.690 ABS at the highest peak, over the 10 hours there was a 0.09 deviation in ABS, the first 5 hours of the run, the sample showed no noticeable decay and was stable. After the ten hours expired, the spectrometer was quickly restarted for another 10 hours, were after the first 4 hours and 20 min in the second run is where the sample reached its half life. Once the 20 hours expired, the ABS reading was 0.082 were it took another 10 hours for Ozone too fully decay.

After 30 days, the sample that was placed in the freezer was removed out of the freezer, with in 5 min the sample was completely thawed, which then a 0.01 ml sample was quickly placed it into the spectrometer were 3 consecutive single non timed scans were taken, with an average highest ABS reading of 0.688. The sample was at full concentration as my initial study 30 days earlier, ozone was fully stabilized, and the frozen state completely eliminated ozone's decay.

In the first PFC experiment, the ozone was stable within the PFC matrix for over 5 hours without any appreciable decay, after 5 hours the sample started to deviate and when the 10 hours mark was reached, the reading was around 0.600 ABS, in the last 5 hours of the run is when the sample lost 0.09 ABS, this was excellent result. In the second part of the experiment is where the sample decay accelerated. Approximately 4 hours and 20 min for the sample to reach its half life, so total time to reach the half life was 14 hours and 20 min. This was excellent result but can be significantly improved, Possible error's that significantly affects time of stabilization after the first 10 hour run, was the seal of the cuvette, by no means was it air tight, ozone was diffusing out, which effected results significantly, also the 260 nm wave length used to detect ozone molecules, that particular wave length that destroys ozone, if readings were taken at timed intervals at every hour instead of continuously, would have yield a better result, but would have been tedious, the temp inside the spectrometer was over the 98 degree F., the room temp that day was 86.5 degrees, a temperature that is not conducive to ozone stabilization, and will accelerates ozone destruction. It should be noted the sample of PFC was 95% pure, and was the only purity I could attain at the time for the experiment and due to time constraints another sample could not have been attained. It should also be noted when the sample was finally removed from the machine for clean up, about 60 hours later after the initial experiment, half the sample evaporated. All and all these experiments are more I can ever hoped for, these key observations proved EMODs where stabilized with in a PFC matrix, in frozen and non frozen states. The utility of this patent is self evident; this invention solves the problems that plagued modern ozone therapy for the last 100 years.

### EXAMPLE 1

### Preparation of a Flavonoid Emulsion

Commercial quercetin was dissolved in 20 ml ethanol at a concentration of 100 mg/ml; a syringe was filled with the prepared solution and quickly injected at a fixed flow rate (2 to 8 ml/min) into supercritical Cot anti-solvent, water, under magnetic stirring (300 to 1000 rpm). Solvent to anti-solvent ratios used were 1:125. The quercetin nano crystal particles were filtered and vacuum dried. Where the nano quercetin crystals were added to 1 g of purified lecithin with 6.8 ml of cold tyrode electrolyte solution, where the glucose in the standard electrolyte tyrode solution is replaced by 2-deoxy-D-glucose (2dg), (ph6.9). The mixture was sonicated for about 20 sec, where it was repeated after 50 sec to 60 sec. At a temp of 4 degree C., 5 ml degassed perfluorodecalin (C10F18) was added and sonicated for 10 periods of 20 sec at 1 min intervals. The emulsion was centrifuged at 4 degree C. for 1 hour at 100 g to sediment large particles. The bottom 5% was discarded. The emulsion contained 35-45% (v/v) dispersed perfluoro-compound, and its pH was between 6.8 and 7.5, the mixture was heat sterilized in an auto clave at 120 c for 6 min. The mixture is ready to add oxygen to auto oxidize the flavonoids and stored at 4 degrees C. Or a mixture of ozone/oxygen may be added by bubbling for 15 min and the emulsion then placed on a bed of dry ice and ethanol mixture to rapidly cool the sample, than placed in a freezer for short term storage. Or a therapeutic mixture ozone/oxygen may be added simultaneously at the time of infusion in a therapeutic setting.

### EXAMPLE 2

A 40% weight per volume of C10f18 flavonoid emulsion was prepared using the method described above in Example 1, having 6% weight per volume lecithin as the emulsifying agent, 0.01% weight per volume tocopherol, 2% weight per volume 2dg as the osmotic agent, and having as a buffer, monobasic sodium phosphate at 0.012% w/v and sodium dichloroacetate at 0.0563% w/v. The emulsion was formulated in accordance with the procedure described above.

### EXAMPLE 3

Where a 100 ml of C10F18 was heat sterilized and degassed, where the PFC was placed in a vacuum atmospheres Glove Box assembly model number He113, the temp was maintained at 6 Degree C under a 80/20 percent mix of oxygen/ozone atmosphere. Where Ozone/oxygen was pump in from a Custom PEM ozone generator that was plumed into the glove box to a glass bubbler for 45 min, Were 50 sealed uni-life prefilled safety syringes within the vacuum glove box were pre-filled and ready to use for ligaments prolo-therapy treatments, which after, the pre filled syringes were removed from the Glove box and placed into a freezer at −85 C for short term storage before shipment in Dry Ice to veterinary clinics.

### EXAMPLE 4

Were a 1 ml sample of oxygenated C10f18 was placed in a Excimer UV light chamber with 222 nm wave length light, and left in the chamber for 5 min, the absorbance was measured in a hatch DR 4000 U spectrometer, and was 0.720 ABS, which after the sample was used to treat and disinfect a cut on a 7 year old Maltese patient.

In closing my invention solves the solubility and stability problems associated in modern ozone therapy but goes beyond just ozone therapy, these reactions described in this invention cannot be performed in another medium. The inertness of PFCs lets us isolate and store reaction intermediates for extended periods. The utility of this invention is evident, and with further study will surely lead to improved therapies to better the human condition.

### References

### McElroy, M. B.; Sze, N. D.; Logan, J. A.; and Ko, M. K., Potential Atmospheric Impact of Explosive Vapor Taggant Molecules, Prepared for the Aerospace Corporation, Washington D.C., January, 1979, NTIS # PB81187189

### Ko, M. K.; Sze, N. D.; McElroy, M. B.; and Wofsky, S. C., Potential Atmospheric Impact of Explosive Vapor Taggant Perfluorohexyl Sulfur Pentafluoride, Prepared for the Aerospace Corporation, Washington D.C., January, 1979, NTIS # PB81-156090 3.

Calloway, A. R.; Stamps, M. A.; and Loper, G. L., Vacuum Ultraviolet and Ultraviolet Absorption Spectra of Various Candidate Vapor Taggants for Blasting Caps. Prepared by the Aerospace Corporation for the U.S. Dept. of the Treasury, March, 1979, NTIS # PB-81-15531 9.
Cancer Res 2006; 66:4410-4418. Published online Apr. 17, 2006. The Use of Ozone in Medicine Mechanisms of Action Munich May 23-25, 2003 Renate Viebahn-Hänsler
Ackey D, Walton T E. Liquid-phase study of ozone inactivation of Venezuelan Equine Encephalomyelitis virus. Appl Environ Microbiol 1985; 50:882-886
Armstrong. Infectious Diseases, First Ed. Mosby, Philadelphia, 2000
Babior B M. Phagocytes and oxidative stress. Am J Med 2000; 109:33-44
Bocci V. Ozone: A New Medical Drug. Springer, 2005
Bocci V. Oxygen-Ozone Therapy: A Critical Evaluation. Kluwer Academic Publishers, Dordrecht, 2002
Bolton D C, Zee Y C, Osebold J W. The biological effects of ozone on representative members of five groups of animal viruses. Environmental Research 1982; 27:476-48
Cann A J. Principles of Molecular Virology, Second Edition. Academic Press, New York, 1997 Cardile V, et al. Effects of ozone on some biological activities of cells in vitro. Cell Biology and Toxicology 1995 Feb; 11(1): 11-21
Carpendale M T, Freeberg J K. Ozone inactivates HIV at noncytotoxic concentrations. Antiviral Research 1991; 16:281-292
Cech T. RNA as an enzyme. Scientific American 1986 Nov; 255(5): 64-76
Clamann H. Physical and medical aspects of ozone. In: Physics and Medicine of the Atmosphere and Space. John Wiley and Sons, New York, 1960, p 151
Dailey J F. Blood. Medical Consulting Group, Arlington Mass., 1998
Dawson T M, et al. Gases as biological messengers: Nitric oxide and carbon monoxide in the brain. J Neuroscience 1994; 14:5147-5159
Delves P J, Roitt I M. Encyclopedia of Immunology. Academic Press: San Diego, 1998
Di Paolo N. Extracorporeal blood oxygenation and ozonation (EBOO) in man. Preliminary report. Int J Artif Organs 01 Feb 2000; 23(2): 131-141
Dulak J, Jozkowicz A. Carbon monoxide: A “new” gaseous modulator of gene expression. Acta Biochimica Polonica 2003; 50(1): 31-47 Dyas A, Boughton B, Das B. Ozone killing action against bacterial and fungal species: Microbiological testing of a domestic ozone generator. J Clin Pathol (Lond) 1983; 36(10): 1102-1104

### Evans A S, Kaslow R A (Eds). Viral Infections in Humans: Epidemiology and Control, Fourth Edition, Plenum, New York, 1997

Gumulka J, Smith L. Ozonation of cholesterol. J Am Chem Soc 1983; 105(7): 1972-1979
Hurst C J. Viral Ecology. Academic Press, New York, 2000
Ignarro L J (Ed). Nitric Oxide: Biology and Pathobiology. Academic Press, 2000
Ishizaki K, Sawadaishi D, Miura K, Shinriki N. Effect of ozone on plasmid DNA of Escherichia coli in situ. Water Res 1987; 21(7): 823-828
Ivanova O, Bogdanov M, Kazantseva V, et al. Ozone inactivation of enteroviruses in sewage. Vopr Virusol 1983; 0(6): 693-698
Knipe D M, Howley P M. Fundamental Virology, Fourth Edition. Lippincott Williams & Wilkins, Philadelphia, 2001
Laskin J D, Laskin D L. Cellular and Molecular Biology of Nitric Oxide. Marcel Dekker, 1999
Lincoln J, Hoyle C H, Burnstock G. Nitric Oxide in Heath and Disease. Cambridge University Press, 1997
Lohr A, Gratzek J. Bactericidal and paraciticidal effects of an activated air oxidant in a closed aquatic system. J Aquaric Aquat 200 East 33rd Street, # 26J New York, N.Y. 10016-4831 Tel. 212-6790679 Fax 212-6798008 Sci 1984; 4(41/2): 1-8
Matus V, Nikava A, Prakopava Z, Konyew S. Effect of ozone on survivability of Candida utilis cells. Vyestsi AkadNavuk Bssr Syer Biyal Navuk 1981; 0(3): 49-52
Matus V, Lyskova T, Sergienko I, Kustova A, Grigortsevich T, Konev V. Fungi; growth and sporulation after a single Treatment of spores with ozone
Max J. Antibodies kill by producing ozone. Science 15 Nov. 2002; 298: 1319
Mudd J B, Leavitt R, Ongun A, McManus T. Reaction of ozone with amino acids and proteins. Atmos Environ 1969; 3:669-682
Menzel D. Ozone: An overview of its toxicity in man and animals. Toxicol and Environ Health 1984; 13:183-204
Olinescu R, Smith T L. Free Radicals in Medicine. Nova Science Publishers, Inc. Huntington, N.Y., 2002
Paulesu L, Luzzi L, Bocci V. Studies on the biological effects of ozone: Induction of tumor necrosis factor (TNF-alpha) on human leucocytes. Lymphokine Cytokine Research 1991; 5:409-412
Razumovskii S D, Zaikov G E. Ozone and Its Reactions With Organic Compounds. Elsevier, New York, 1984
Rilling S, Veibahn R. The Use of Ozone in Medicine. Haug, New York, 1987
Rilling S. The basic clinical applications of ozone therapy. Ozonachrichten 1985; 4:7-17 Smith L L. Cholesterol autoxidation of lipids. Chemistry and Physics of Lipids. 1987; 44:87-125 Snyder S. Drugs and the Brain. Scientific American Library Series, 1996
Rice R G. Century 21—Pregnant with ozone. Ozone Science and Engineering 2002; 24: 1-15
Riesser V, Perrich J, Silver B, McCammon J. Possible mechanism of poliovirus inactivation by ozone. In: Forum on Ozone Disinfection. Proceedings of the International Ozone Institute. Syracuse, N.Y., 1977:186-192
Roy D, Wong P K, Engelbrecht R S, Chian E S. Mechanism of enteroviral inactivation by ozone. Appl Envir Microbiol 1981; 41:718-723
Roy D, Engelbrecht R S, Chian E S: Comparative inactivation of six enteroviruses by ozone. Am Water Works Assoc J 1982; 74(12): 660-664
Sunnen G. Ozone in Medicine. Journal of Advancement in Medicine. 1988 Fall; 1(3): 159-174
Sunnen G. Possible mechanisms of viral inactivation by ozone. Townsend Letter for Doctors. Ap 1994: 336
Sweet J, Kao M S, Lee D, Hagar W. Ozone selectively inhibits growth of human cancer cells. Science 1980; 209:931-933
Thanomsub B. Effects of ozone treatment on cell growth and ultrastructural changes in bacteria. J Gen Appl Microbiol 1 Aug. 2002; 48(4): 193-199
Valentine G S, Foote C S, Greenberg A, Liebman J F (Eds). Active Oxygen in Biochemistry. Blackie Academic and Professional, London, 1995
Vaughn J M, Chen Y, Linburg K, Morales D. Inactivation of human and simian rotaviruses by ozone. Applied Environmental Microbiology 1987; 48:2218-2221
Viebahn R. The Use of Ozone in Medicine. Odrei Publishers, Iffezheim, 1999
Wells K H, Latino J, Gavalchin J, Poiesz B J. Inactivation of human immunodeficiency virus Type 1 by ozone in vitro. Blood 1991 Oct; 78(7): 1882-1890
Wentworth P, McDunn J E, Wentworth A D, et al., Evidence for antibody-catalysed ozone formation in bacterial killing and inflammation. Science 13 Dec 2002; 298: 2195-2199
Wink D A, Grisham M B, Mitchell J B, Ford P C. Direct and indirect effects of nitric oxide in chemical reactions relevant to biology. Methods Enzymol 1996; 268:12-31
Wolcott J, Zee Y C, Osebold J. Exposure to ozone reduces influenza disease severity and alters distribution of influenza viral antigens in murine lungs. Appl Environ Microbiol 1982; 443:723-731
Yu B P. Cellular defenses against damage from reactive oxygen species. Physiological Reviews 1994 Jan; 74(1): 139-

1. Wherein a heat sterilized highly fluorinated bio compatible fluorocarbon comprises of a stabilized free radical suspension, where the highly fluorinated fluorocarbon free radical suspension comprises of a liquid Fluorocarbon continuous phase, where oxygen, electronically modified oxygen derivatives (EMODs), and or electronically modified reaction intermediates, is suspended within the matrix of the PFC solution, where said the highly fluorinated fluorocarbon is used as an inert medium to stabilize electronically modified oxygen derivatives and or reaction intermediates in a combination thereof, for the purpose of delivering to mammalian patient in a concentration that is therapeutic to induce a cascading immune response.

2. Method to claim 1. Wherein a heat sterilized highly fluorinated fluorocarbon can be further made into a emulsion, were the fluorocarbon emulsion comprises of a liquid aqueous continuous phase, a discontinues fluorocarbon phase, having suspended oxygen, electronically modified oxygen derivatives, reaction intermediates, benzo-γ-pyrone derivatives, emulsifying agents, phospholipids, egg lecithin, soy lecithin, glucose, glucose derivatives, buffers, electrolytes, pro-oxidants, bio-active agents, glycolic inhibitors, thioredoxin inhibitors, electron chain blockers, antioxidants, vitamins, these components of the fluorocarbon emulsion including the aqueous solution is to be used together, separate or in a combination thereof.

3. Method to claim 1. Where the Fluorocarbon suspension in this invention is in a form, selected from a group, consisting of liquids, foams, creams, solids, slurries, dispersions, sols, emulsion, miscalls, gels, micro emulations, reverse emulations, or in combination of thereof.

4. Method to claim 1. Wherein EMODs are created externally and delivered to the fluorocarbon solution or a fluorocarbon emulsion, through bubbling ozone/oxygen mix through the solution, or ozone/oxygen gas is injected under pressure, under a partial vacuum, fully evacuated system or a combination thereof.

5. Method to claim 1. Wherein EMODs and or reaction intermediates are created within the oxygenated fluorocarbon solution or oxygenated fluorocarbon emulsion through UV radian, ultra sonic cavitations, magnetic fields, radiation, laser light, high energy particles, or in combination thereof.

6. Method to claim 1. Wherein electronic modified derivatives is created through a catalytic reaction in the fluorocarbon solution or fluorocarbon emulsion, where the catalyst are active metals from the periodic chart or enzymes.

7. Method to claim 1. Where the fluorocarbon suspension in this invention, is delivered to a mammalian body, intravenously, subcutaneously, intramuscularly, topically, parenteral, intracavitary, or in a combination thereof.

8. Wherein the fluorocarbon in this invention according to claim 1, is used as a dielectric with an applied voltage to drive redox reactions within the PFC matrix.

9. Where all Physiological compounds can be activated or electrically modified within the PFC matrix of claim 1, using a physiological gas, catalytic reaction, using an applied voltage were the PFC is used as the dielectric, or a combination thereof.

10. Method to claim 1. Wherein electronically modified reactive intermediates in this Invention is stabilized in an inert PFC matrix, at room temperatures, low temperatures, frozen or cryogenically frozen, where the free radical PFC solution is to be used immediately after creation or stored in a frozen state for later use.

11. Method to claim 2. compounds to be suspended within the pfc matrix are selected from a group, but not limited to the group of, simple phenols, polyphenols, benzoquinones, phenolic acid, phenyacetic, acid, cinnamic acid, alpha lipolic acid, selininite, turtbuyl, catechins, chalcones, ligins, phenylpropenes, coumarins, chromones, naphthoquinones, xanthones, stilbenes, anthraquinones, xanthones, glycosides, Saponin, flavonoids, flavones, flavonols, flavanonols, flavanones, flavanone glycoside, flavanols catechins, lecithins, egg yolk, polyoxyethylene-polyoxypropylene copolymers, sorbitan polyoxy-ethylenes, phospholipids, soy or synthetic lipids, perfluoroalkyl phospholipid, perfluoroalkyl surfactants, chalcones, ligins, sodium dichloroacetate, potassium dichloroacetate, and diisoproyl ammonium dichloroacetate, dichloroacetic acid, anthocyanidins, isoflavones, flavonol glycosides, bifflavoniods, peroxides, Quinone methides, semi quinines, O-quinone, hydroxyl compounds, carboxyl, 2-turbuty compounds, iso alky compounds, robustaflavone, hinokiflavone, amentoflavone, agathisflavone, volkensiflavone, morelloflavone, rhusflavanone, succedaneaflavanone, Antiviral fiflavanoid derivatives and salt forms thereof, e.g. robustaflavone tetrasulfate potassium salt, In a combination thereof.

12. Method to claim 2. Were the thioredoxin inhibitor and super oxide radical generator in vivo are benzo-γ-pyrone derivatives with A, B, and C rings, such as, Quercetin, Myricetin, Fisetin, reservatrol.

13. Method to claim 2. Wherein the osmotic agent for this invention can be any sugar or sugar derivative but preferable compound's to be selected are, hexa-hydric alcohol such as mannitol or sorbitol, or a sugar such as glucose, mannose, glycerol, polyethylene glycol, propylene glycol, fructose, 2-deoxy-D-glucose, 2(DG), 2-Deoxy-2-(18F)fluoro-D-glucose fructose.

14. The fluorocarbon emulsion of claim 2 further comprising a buffering agent selected from the group consisting of, tris(hydroxymethyl)aminomethane, imidazole, sodium bicarbonate, zinc salts, monobasic sodium phosphate, dibasic sodium phosphate, magnesium sulfate, magnesium chloride, sodium chloride, potassium chloride, monobasic potassium phosphate, dibasic potassium phosphate, sodium dichloroacetate, potassium dichloroacetate, and diisoproyl ammonium dichloroacetate, dichloroacetic acid and in combination thereof.

15. Method to claim 1. Wherein the fluorocarbon for this invention is selected from a group consisting of, fluorinated cyclic compounds, fluorinated amines, fluorinated alkanes, fluorinated hydrides, fluorinated alkenes, halogenated fluorocarbons, fluorinated ethers, fluorinated polyether's, fluorinatedamines, derivatives thereof, fluorocarbon compounds may be used alone or in combination thereof.

16. Method to claim 2. Where the fluorocarbon emulsion in this invention is created by ultrasonic cavitation, and or through high-pressure homogenization or in a combination thereof.

17. Wherein the flavonoid benzo-γ-pyrone derivatives with A, B, C rings in this invention are dissolved in a organic solvent and were a super critical anti solvent is used to precipitate nano-crystalline particles that are vacuum dried and stored prior to emulsification.

18. Method to claim 1. Wherein the fluorocarbon suspension in this invention can be used with compounds selected from the group, consisting of anions, cations, antibiotics, anti-inflammatories, zinc compounds, silver compounds, antineoplastics, anesthetics, antiviral, carbon nano particles, gold nano particles, carbon nano matrices, iron oxides, metallic particulates, active metals, all minerals, enzymes, active principals, nucleic acids, genetic material, corticosteroids, immunoactive agents, steroids, viral vectors, florescent agents, fluorinated solids, immunosuppressive agents, peptides, proteins, radioactive particles, RNA, mRNA.

19. Method to claim 2. Where two emulsions are created that are physically separated, one with the oxidant or catalyst and the other having a bio active agent, were the oxidant or catalyst comes in contact with the bio active agent and activates it, at or before infusion in a mammalian patient.

20. Method to claim 1. Wherein the fluorocarbon in this invention can be used with synthetic and non synthetic extra cellular membranes suspended within the PFC matrix.
